(19) United States
(12) Patent Application Publication
(10) Pub. No.: US 2023/0091557 A1
(43) Pub. Date: Mar. 23, 2023

(54) RESPIRATORY ASSIST AND FLUID REMOVAL DEVICE FOR TREATMENT OF RESPIRATORY DISTRESS SYNDROME

(71) Applicants: Kevin K. Chung, Bethesda, MD (US); THE CHARLES STARK DRAPER LABORATORY, INC., Cambridge, MA (US)

(72) Inventors: Jeffrey T. Borenstein, West Roxbury, MA (US); David W. Sutherland, Belmont, MA (US); Kevin K. Chung, Bethesda, MD (US)

(21) Appl. No.: 17/948,738

(22) Filed: Sep. 20, 2022

Related U.S. Application Data
(60) Provisional application No. 63/246,117, filed on Sep. 20, 2021.

(51) Int. Cl. A61M 1/16; A61M 1/36

(52) U.S. Cl. CPC A61M 1/1698; A61M 1/3666; A61M 1/1603; A61M 1/1623; A61M 1/3673; A61M 2205/3331

(57) ABSTRACT

An extracorporeal blood treatment module includes a plurality of gas transfer units, having a first polymer layer with a plurality of gas channels, a second polymer layer with a plurality of blood channels, and a gas permeable membrane disposed between the plurality of gas channels and the plurality of blood channels, a fluid transfer unit integrated with the plurality of gas transfer units, and including a third polymer layer having a plurality of fluid collection channels, a fourth polymer layer having a plurality of blood channels, and a fluid permeable membrane disposed between the plurality of fluid collection channels and the plurality of blood channels, and a housing containing the plurality of gas transfer units and fluid transfer unit.

### CROSS-REFERENCE TO RELATED APPLICATIONS

This application claims priority under 35 U.S.C. § 119(e) to U.S. Provisional Patent Application Ser. No. 63/246,117, titled “RESPIRATORY ASSIST AND FLUID REMOVAL DEVICE FOR TREATMENT OF RESPIRATORY DISTRESS SYNDROME,” filed Sep. 20, 2021, the entire contents of which is incorporated herein by reference for all purposes.

### FIELD

Aspects and examples disclosed herein are generally directed to microfluidic platforms that enable integration, simplification, and improved safety and efficiency, with blood oxygenation and fluid removal in a single device.

### SUMMARY

In accordance with on aspect, there is provided an extracorporeal blood treatment module. The extracorporeal blood treatment module comprises a plurality of gas transfer units, each of the gas transfer units including a first polymer layer having a plurality of gas channels, a second polymer layer having a plurality of blood channels, and a gas permeable membrane disposed between the plurality of gas channels and the plurality of blood channels and providing for the transport of gas between the plurality of gas channels and the plurality of blood channels, a fluid transfer unit adhered to at least one of the plurality of gas transfer units, the fluid transfer unit including a third polymer layer having a plurality of fluid collection channels, a fourth polymer layer having a plurality of blood channels, and a fluid permeable membrane disposed between the plurality of fluid collection channels and the plurality of blood channels and providing for the transport of fluid from the plurality of blood channels into the plurality of fluid collection channels while preventing transport of blood cells from the plurality of blood channels into the plurality of fluid collection channels, and a housing containing the plurality of gas transfer units and fluid transfer unit.

In some examples, the blood treatment module further comprises a blood inlet manifold having channels fluidically coupled to each of the plurality of blood channels in both the plurality of gas transfer units and the fluid transfer unit.

In some examples, the blood treatment module further comprises a blood exit manifold having channels fluidically coupled to each of the plurality of blood channels in both the plurality of gas transfer units and the fluid transfer unit.

In some examples, the blood treatment module further comprises a fluid exit manifold fluidically coupled to each of the plurality of fluid collection channels in the fluid transfer unit.

In some examples, the blood treatment module further comprises a pump fluidically coupled to the fluid exit manifold and configured to create a transmembrane pressure across the fluid permeable membrane.

In some examples, the blood treatment module further comprises a pressure regulator fluidly coupled to the pump and fluid exit manifold and configured to maintain the transmembrane pressure at a desired level.

In some examples, the blood treatment module further comprises a transmembrane pressure sensor configured to measure the transmembrane pressure.

In some examples, the blood treatment module further comprises a controller configured to receive transmembrane pressure measurements from the transmembrane pressure sensor and adjust operation of one of the pump or the pressure regulator based on the transmembrane pressure measurements.

In some examples, an upper surface of the third polymer layer is bonded to a lower surface of the second polymer layer of one of the plurality of gas transfer units.

In some examples, the one of the plurality of gas transfer units is at an end of a stack of the plurality of gas transfer units.

In some examples, a lower surface of the fourth polymer layer is bonded to an upper surface of the first polymer layer of one of the plurality of gas transfer units.

In some examples, the one of the plurality of gas transfer units is at an end of a stack of the plurality of gas transfer units.

In some examples, the plurality of blood channels in each of the plurality of gas transfer units are oriented perpendicular to the plurality of gas channels.

In some examples, the plurality of blood channels in the fluid transfer unit are oriented perpendicular to the plurality of fluid collection channels.

In some examples, surfaces of the blood channels in each of the plurality of gas transfer units and in the fluid transfer unit are coated with an anticoagulant.

In some examples, the blood treatment module is configured to treat up to seven liters of blood per minute in the plurality of gas transfer units.

In some examples, the blood treatment module is configured to raise O₂ₛₐₜ of blood passing through the plurality of gas transfer units from 75% or lower to 95% or higher in a single pass.

In some examples, the blood treatment module is configured to treat up to 100 mL of blood per minute in the fluid transfer unit.

In some examples, the blood treatment module is configured to remove up to seven liters of fluid per day from blood passing through the fluid transfer unit.

In some examples, the blood channels of the plurality of gas transfer units are substantially identical in size and shape to the blood channels of the fluid transfer unit.

In some examples, the gas channels of the plurality of gas transfer units are substantially identical in size and shape to the fluid collection channels of the fluid transfer unit.

In accordance with another aspect, there is provided a blood treatment module comprising a first plurality of polymer layers each having a plurality of blood channels; and a second plurality of polymer layers each having a one of a plurality of gas channels or fluid collection channels, each of the first plurality of polymer layers secured to at least one of the second plurality of polymer layers with a membrane, a plurality of the membranes being gas permeable membranes, at least one of the membranes being a fluid permeable membrane.

In accordance with another aspect, there is provided a method of treating blood. The method comprises passing the blood through any examples of the blood treatment modules described above.

### DESCRIPTION OF THE DRAWINGS

The accompanying drawings are not intended to be drawn to scale. For purposes of clarity, not every component may be labeled in the drawings.

In the drawings:

FIG. 1A is an exploded isometric view of a number of blood treatment units and blood inlet and outlet manifolds of an example of a blood treatment module;

FIG. 1B is a block diagram illustrating components of an example of a blood treatment module;

FIG. 2A shows a CNC-machined master mold for a layer material including channels for blood, air, or excess fluid in examples of blood treatment modules disclosed herein;

FIG. 2B illustrates a cast silicone layer in the mold of FIG. 2A;

FIG. 2C illustrates an assembled oxygenator layer for examples of blood treatment modules disclosed herein;

FIG. 2D illustrates an SEM cross-section of the oxygen channel-membrane-blood channel sandwich structure for examples of blood treatment modules disclosed herein;

FIG. 3A shows an SEM of a junction region in a layer material including channels for blood, air, or excess fluid in examples of blood treatment modules disclosed herein where the channel depth varies smoothly;

FIG. 3B shows a SEM of a smoothly branching channel junction region in a layer material including channels for blood, air, or excess fluid in examples of blood treatment modules disclosed herein;

FIG. 4 illustrates the fabrication process for blood oxygenation fluidic units for examples of blood treatment modules disclosed herein;

FIG. 5 illustrates the fabrication process for excess fluid removal units for examples of blood treatment modules disclosed herein;

FIG. 6 illustrates filtrate removal rate Q vs. transmembrane pressure TMP;

FIG. 7 is a chart showing results of testing of volume percent oxygen transfer in a blood oxygenation component of a device as disclosed herein as a function of the ultrafiltration rate drawn from a renal replacement therapy component the device;

FIG. 8 is a chart showing ultrafiltration rate vs. transmembrane pressure experimental data from a renal replacement therapy component of a device as disclosed herein; and

FIG. 9 is a chart of measured hematocrit change as a function of ultrafiltration rate in a device as disclosed herein.

### DETAILED DESCRIPTION

Two of the most essential organ support systems administered in critical care settings are respiratory assist and fluid removal. The former is administered to patients suffering from ARDS (Acute Respiratory Distress Syndrome) or related conditions that compromise the ability of the lungs to adequately oxygenate or remove carbon dioxide from a patient's blood, and includes various forms of acute lung injury arising from severe infections, drowning, aspiration, traumatic injury, and exacerbations of chronic lung diseases such as Chronic Obstructive Pulmonary Disease (COPD). Many of these occurrences are accompanied by hypervolemia, or fluid overload, which is often the proximate cause of ARDS, and is also associated with acute renal failure (ARF), sepsis, and related conditions. Currently, patients may receive one or more treatment modalities among ExtraCorporeal Membrane Oxygenation (ECMO) to oxygenate the blood, ExtraCorporeal CO₂ Removal (ECCO2R) to remove carbon dioxide from the blood, hemofiltration/ultrafiltration to remove excess fluid, hemodialysis to remove waste products from the blood, and hemoadsorption to remove excess cytokines from the blood arising from sepsis. Other related conditions such as liver failure require more complex procedures.

Currently, patients suffering from respiratory failure may be treated with non-invasive or invasive mechanical ventilation (MV). In particularly severe cases, or when respiratory failure is refractory to treatment with MV, patients may be placed on ECMO in advanced care settings, or in specific cases such as battlefield-related traumatic injuries or infections, prolonged field care or medevac scenarios. In addition to ECMO, patients may be treated with hemofiltration for hypervolemia and/or hemodialysis for ARF and/or hemoadsorption for sepsis, depending on the trajectory and severity/complexity of their disease. In instances in which multiple of these functions are required to be administered to a critically ill patient, the clinical team is forced to either separately treat the patient with each of the necessary therapies, or to find an engineered solution that brings multiple extracorporeal therapies together in a complex ganged circuit configuration. Often this entails multiple vascular access points, multiple blood pumps, multiple anticoagulation circuits and bubble/occlusion traps, and lengthy and complex tubing sets and circuit paths. For anywhere but the most advanced clinical centers, this represents an extremely difficult challenge.

In spite of the prevalence of acute kidney injury in respiratory failure patients, efforts to combine renal replacement therapy with ECMO remain difficult. In addition to many logistical and operational challenges, a fundamental barrier to integration is caused by differences in the configuration of hollow fiber dialyzers (HFD) versus hollow fiber membrane oxygenators (HFMO.) For HFD, blood flows intra-luminally with filtrate/dialysate contained in the extraluminal space, while for ECMO cartridges, blood flows extraluminally and oxygen flows through the fiber lumens, and ECMO blood flow rates generally exceed those of renal replacement therapy (RRT) by an order of magnitude, creating an asymmetry in the operational requirements for each component of a multi-organ system. Therefore, a means to realize blood oxygenation and fluid removal in a single device/circuit represents a significant advance.

Aspects and examples disclosed herein provide the critical care team with a single device, of approximately the same level of complexity as the ECMO/respiratory assist circuit, that achieves both blood oxygenation and excess fluid, for example, water removal. With hardly any additional effort or operational complexity, two of the most critical and challenging organ support functions are combined into one treatment/device.

Microfluidic organ assist device technologies present an opportunity to combine RRT and ECMO into a single monolithic cartridge, since blood flows within microchannel lumens in each device configuration. For microfluidic RRT, blood is separated from a filtrate chamber by a sandwiched porous membrane, while in microfluidic ECMO, a solid gas permeable membrane separates the blood and carrier gas channels. Therefore, it is possible to create a single cartridge with multiple layers stacked and integrated via entry and exit manifolds, with one or more layers comprising the ECMO function while others provide the RRT function. Given the asymmetry in blood flow rate requirements for the ECMO and RRT components, a single monolithic cartridge may include a stack of many microfluidic ECMO layers with one RRT layer inserted, all joined by entry and exit blood distribution manifolds in a seamless manner that is operationally no more complex than an individual ECMO or RRT cartridge.

Aspects and examples disclosed herein include a high hemocompatibility full-scale oxygenator for respiratory support in prolonged field care (PFC), combined with an integrated fluid management component that provides two critical care functions in one streamlined device. Aspects and examples disclosed herein leverage progress in the development of microfluidic organ assist device technologies toward an integrated platform capable of oxygenating the blood and managing fluid balance in a single extracorporeal device. As described herein, the design of these functional elements of the device is based upon physiological principles of blood flow, to provide a design that flows blood smoothly through a microchannel network. The combination of biomimetic branching blood flow patterns, highly efficient oxygenation and fluid removal designs, in an integrated respiratory assist and ultrafiltration (UF) functional unit, optionally treated with anticoagulant coatings on all surfaces that blood would contact during use, will markedly improve patient outcomes in PFC. Microfluidics technology enables seamless integration of respiratory support and fluid management in one cartridge, a major simplification of patient treatment. This is possible because in both the microfluidic oxygenator (ECMO) and UF system, blood flows through microchannels in a bonded circuit, while in conventional hollow-fiber-based systems, blood flows around the oxygenator fibers and then must transition into a second system where blood flows through the fibers while filtrate is removed from the interstitial space between the fibers.

Aspects and examples disclosed herein leverage an approach toward microfluidic oxygenator devices that utilizes precision-controlled computer numerical control (CNC) machining tools to fashion fully three-dimensional networks with smoothly varying cross sections, creating metal master molds from which highly gas permeable transparent silicone (for example, PDMS) layers can be cast thousands of times. These silicone layers, patterned with either blood channel or gas channel networks, are bonded together with an intervening thin gas transfer membrane, and layers are stacked into a three-dimensional network joined by blood distribution manifolds designed to distribute blood uniformly and to control fluid shear within a desirable physiological range. Precision machining enables blood channels to be joined smoothly and gradually, avoiding sharp corners and sudden transition regions known to disturb blood flow streamlines. Larger trunk lines have greater channel depth, making efficient use of space and avoiding extremely wide channels to accommodate larger flows in entry and exit regions. Vertical blood distribution manifolds that join layers may be fabricated from directly machined transparent hard plastics, with rounded channel cross-sections and smoothly varying dimensions that carry blood to and from the extracorporeal circuit.

Each of the polymer layers of the plurality of blood oxygenation/CO₂ removal (ECMO) fluidic units are stacked upon one another such that the channels in a first polymer layer substantially overlap and run perpendicular to the channels of polymer layers on either side of the first polymer layer. In some implementations, the plurality of ECMO fluidic units includes between 10 and 100, between 30 and 80, or between 40 and 60 or more stacked polymer layers. In some implementations, the polymer layers are manufactured from materials and with thickness selected to provide an oxygen gas permeance greater than about 1×10⁻⁶ mL/s/cm²/cm Hg, about 1×10⁻⁵ mL/s/cm²/cm Hg, about 3×10⁻⁵ mL/s/cm²/cm Hg, about 7×10⁻⁵ mL/s/cm²/cm Hg, or greater than about 1×10⁻⁵ mL/s/cm²/cm Hg. In some implementations, the material(s) and thickness of gas permeable material separating gas flow channels from adjacent blood flow channels provides for a carbon dioxide gas permeance between the gas and blood flow channels of at least 1×10⁻⁶ mL/s/cm²/cm Hg, 1×10⁻⁵ mL/s/cm²/cm Hg, 2×10⁻⁵ mL/s/cm²/cm Hg, or 5×10⁻⁵ mL/s/cm²/cm Hg.

In some implementations, the polymer layers are manufactured from Poly(DiMethylSiloxane) (PDMS), PDMS variants such as MDX-4, and modified PDMS compositions that enhance gas (e.g., oxygen and carbon dioxide permeability), polyethylene, or polyurethane. One example material of which the polymer layers may be formed is Nusil MED-6015 silicone elastomer two-part polymer (Avantor, Radnor, Pa.). The polymer layers may be directly stacked upon one another. For example, when the blood and gas channels of the polymer layers are defined within a PDMS layer, oxygen can saturate from the gas channels and into the PDMS. The PDMS then serves as a source of oxygen for the blood channels displaced vertically and aligned perpendicular to the gas channels. In some implementations, manufacturing the polymer layers from materials with a high permeability to oxygen enables the polymer layers to be directly stacked on one another without the need of a gas permeable membrane between the polymer layers. In some implementations, the gas channels and the blood channels are separated from one another (out-of-plane) by between about 25 μm and about 200 μm, between about 25 μm and about 150 μm, between about 25 μm and about 100 μm, between about 25 μm and about 75 μm, or less than 25 μm.

In other implementations, the polymer layers are manufactured from thermoplastics, such as polystyrene, polycarbonate, polyimide, or cyclic olefin copolymer (COC), biodegradable polyesters, such as polycaprolactone (PCL), or soft elastomers such as polyglycerol sebacate (PGS). In these implementations, each of the polymer layers are separated from one another by a gas permeable membrane selected to permit diffusion of oxygen or other gas between the blood channels and the gas channels, for example, a PDMS membrane having a thickness of, for example, about 50 μm or less. One suitable material from which the gas permeable membrane may be formed is 50 μm-thick Silpuran® gas permeable silicone membrane (Wacker, Munich, Germany). The gas permeable membranes may be non-porous to prevent passage of liquids from the blood channels into the gas channels.

In other embodiments, the polymer layers and gas permeable membrane may both be formed of a material such as silicone, either the same or different grade or formulation.

In some implementations, having the polymer layer act as a gas source reduces the alignment tolerance needed to construct the ECMO layer stack. For example, as oxygen passes through the gas channels the oxygen can saturate the polymer layers and act as a gas source to the blood channels even if the gas channels are not perfectly aligned with the blood channels. In devices where a membrane separates two non-gas permeable channel containing layers, diffusion may substantially only occur through the membrane at locations where gas and blood channels overlap. In some implementations of the ECMO fluidic units described herein, because a substantial portion of the polymer layer acts as a gas source to the blood channels rather than just the portion of the membrane at overlapping areas of gas and blood channels, the alignment and overlap tolerances of the gas and blood channels can be lower in the devices described herein than compared to membrane based devices. Therefore, in some implementations, the gas channels and the blood channels in different polymer layers of the devices described herein can be at least partially offset from one another without departing from the scope of the invention.

Each of the gas channels in the gas channel polymer layers and the blood channels in the blood channel polymer layers are defined as troughs in surfaces of the polymer layers. The troughs define the sidewalls and the floor of the gas channels and blood channels. The ceiling of each of the channels is provided by a bottom surface of a polymer layer that is stacked upon the surface of the polymer layer that defines the troughs. In some implementations, a top layer that does not include any channels provides the ceiling for the channels defined in a polymer layer. Each of the gas channels in a gas channel polymer layer may run parallel to other gas channels in the gas channel polymer layer along a majority of the length of the gas channel. Each of the blood channels in a blood channel polymer layer may run parallel to other blood channels in the channel polymer layer along a majority of the length of the blood channel.

In some implementations, the polymer layers of the ECMO fluidic units include an alternating channel pattern of polymer layers having gas channels and polymer layers having blood channels, with the blood and gas channels separated from one another by gas permeable membranes. For example, the alternation pattern can include a strictly alternating pattern where each blood channel polymer layer is between two gas channel polymer layers and each gas channel polymer layer is between two blood channel polymer layers (other than at the top and bottom of the stack of polymer layers). In other implementations, the alternation pattern may include multiple gas channel polymer layers or multiple blood channel polymer layers next to one another. For example, the polymer layer stack could include an alternation pattern that includes two gas channel polymer layers, then two fluid channel polymer layers, then two gas channel polymer layers, then two fluid channel polymer layers, and so forth. In some other implementations, the alternation pattern may include multiple blood channel polymer layers alternating with one gas channel polymer layer having a height about equal to the sum of heights of the multiple blood channel polymer layers, followed by another set of blood channel polymer layer. When stacked, each gas channel in a given gas channel polymer layer would be positioned substantially perpendicular with, and under or over, a corresponding set of multiple blood channels in an adjacent blood channel polymer layer. While a variety of alternation patterns can be suitable for the system described herein, the remaining portion of the disclosure assumes a strictly alternation pattern; however, one of ordinary skill in the art would appreciate the systems described herein may be implemented within any alternation pattern.

In some implementations, the blood channels are configured to distribute blood while protecting blood health. For example, the walls of the blood channels can be coated with an anticoagulant to prevent clotting of blood as the blood flows through the blood channels. Also, to protect the health of the blood flowing through the blood channels, the blood channels can include gradual angles rather than right angles. For example, a primary blood input channel may gradually transition into the plurality of blood channels crossing the polymer layer and the plurality of blood channels may gradually transition into a primary blood outlet channel.

In some implementations, the relative dimensions of the blood channels are selected to follow Murray's Law. In some implementations, the blood channels are between about 1 cm and about 40 cm, between about 10 cm and about 30 cm, between about 15 cm and about 25 cm long, or about 15 cm long. The blood channels may be between about 100 μm and about 1000 μm, between about 300 μm and about 800 μm, between about 500 μm and about 600 μm, or about 500 μm wide (across the majority of their lengths). The blood channels may be separated from another by a similar or same distance as their width, for example, the blood channels may have widths of about 500 μm and may be separated from one another in the plane of the polymer layer in which they are formed by about 500 μm. The blood channels may be between about 40 μm and about 250 μm, between about 100 μm and about 200 μm, between about 100 μm and about 150 μm, or about 200 μm deep. The blood channels may have a height to width ratio in the range of 1:1 to about 1:6, or about 1:1 to about 1:3. In certain examples, the blood channels may have height to length ratio in the range of 1:250 to about 1:800, or about 1:250 to about 1:400. In certain examples, the blood channels may have width to length ratio in the range of 1:250 to about 1:800, about 1:250 to about 1:400, or about 1:250 to about 1:1. In some implementations, each ECMO blood channel polymer layer includes between about 5 and about 100, between about 20 and about 80, between about 40 and about 60 blood channels, between about 100 and about 1000, between about 1000 and about 8000 blood channels, or between about 5000 and about 7000 blood channels. In some implementations, each ECMO gas channel polymer layer includes between about 5 and about 100, between about 20 and about 80, between about 40 and about 60 gas channels, between about 100 and about 1000, between about 1000 and about 8000 gas channels, or between about 5000 and about 7000 gas channels. The gas channels may have dimensions within the same ranges as given for the blood channels above.

The blood channels can also be characterized according to the fluid shear rate at the walls of the channels observed as a blood travels through the channels. In certain examples, any blood channel is characterized as having a fluid shear rate in the range of about 100 s⁻¹ to about 4000 s⁻¹ for blood at 37.0° C., a range of about 100 s⁻¹ to about 3000 s⁻¹ for blood at 37.0° C., a range of about 400 s⁻¹ to about 2200 s⁻¹ for blood at 37.0° C., a range of about 1000 s⁻¹ to about 2200 s⁻¹ for blood at 37.0° C., a range of about 1500 s⁻¹ to about 2200 s⁻¹ for blood at 37.0° C., or a range of about 1900 s⁻¹ to about 2200 s⁻¹ for blood at 37.0° C.

It is contemplated that blood channels with various types of cross-sections are amenable for use in the microfluidic devices described herein. For example, in certain instances, the cross-sections of the blood channels may be rectangular, round, triangular, semi-circular, or other geometries. It is contemplated that certain cross section geometries described herein can minimize shear stress experienced by blood traveling through blood channels in the device. For example, it is completed that rounded or semi-circular cross-sectional geometries can minimize shear stress experienced by blood traveling through the blood channels and enhance the surface-to-volume ratio of the blood channels. Accordingly, in certain examples, any blood channel may have a cross-section that is semi-circular.

Further, the cross-section geometry of the blood channels can be selected to minimize the pressure that must be exerted on the blood to force the blood through the microfluidic device. Cross-section geometries that promote fluid transfer through the chamber with minimal friction with the walls of the chamber are completed to minimize the pressure that must be exerted on the blood to force the blood through the microfluidic device. In addition, cross-section geometries that promote fluid transfer by minimizing losses associated with layers of fluid moving through the chambers are completed to minimize the pressure that must be exerted on the fluid to force the fluid through the microfluidic device.

In some implementations, the blood channels include mechanical features that stimulate mixing of the blood as it flows through the blood channels. For example, one or more walls of the blood channels can include pits, posts, ridges, grooves, or a combination thereof that mix the blood as it flows through the blood channels. Structures that induce blood mixing can include topographic features directing blood out of line with the flow direction (such as cross-hatched patterns or ridges placed diagonal to the flow), flexible elements that deform under the flow to create temporal perturbations in the blood, and elements that induce rotational flows within the flow stream. Accordingly, in certain examples, a blood channel further comprises a mixing element to induce fluid mixing. In certain other examples, a blood channel comprises one or more changes in height or width of the channel along the longitudinal axis of the channel.

Another feature of the blood channels relates to two-dimensional structures, such as, networks of branched or bifurcated channels. The networks may feature smooth bifurcations and/or gradual changes in the cross-sectional channel dimensions, and may mimic the physiological properties of in-vivo vascular and/or micro-vascular networks.

The inner surface of blood channels can be modified to achieve certain performance properties, such as improved resistance to degradation caused by a particular substance that may be present in the blood, or reduce the risk that the blood channels may cause a transformation (e.g., inducement of blood clotting) of certain components in the blood. The surface modification may be a partial coating of the inner walls of the blood channels with a particular substance or a complete coating of the inner walls of the blood channels with a particular substance. Surface modifications that alter blood-material interactions can include surface-tethered compounds that reduce clotting (such as heparin), hydrophobic/hydrophilic monolayers that control protein adsorption to the device, degradable coatings that reduce build-up of adsorbed species in the device, and energetic treatments (such as energetic oxygen plasma) that alter surface chemistry and subsequent hydrophobicity/hydrophilicity. In certain examples, any blood channel is coated with a biological molecule, such as serum albumin or a surface protein that can be found in vasculature. In certain examples, any blood channel is coated with an anti-coagulant (such as heparin), which is contemplated to reduce blood clotting in blood channels.

The microfluidic device may comprise a distribution system for delivering gas to any gas channel in the device, and delivering blood to any blood channel in the device. The distribution system may comprise branching or bifurcating microchannels, biomimetic vascular-like channels, or a manifold structure. Controllable access to the chambers may be provided by vascular-like channel structures, structures that provide a smooth path for fluid flow, or other configurations.

Accordingly, in certain examples, the microfluidic device further comprises means for delivering gas to the gas channels and blood to the blood channels. In certain examples, the delivery means bridge large to small conduits.

The microfluidic devices described herein may optionally contain one or more of: (i) a first access conduit affording fluid communication with an input end of one or more blood channels; (ii) a first return conduit affording fluid communication with an output end of one or more blood channels; (iii) a first pump for ensuring that blood entering the first access conduit flows through one or more blood channels and out the first return conduit, (iv) a first access conduit affording fluid communication with an input end of one or more gas channels; and (vi) a second pump for ensuring that a gas entering the first access conduit flows through one or more gas channels.

Access and return conduits can convey blood to and from the blood channels. Access may be through an IV needle, cannula, fistula, catheter, or an implanted access device. The access points may be existing points for previous treatments (e.g., hemodialysis) and may be arterio-venous or veno-venous in nature. The conduits can be standard medical tube materials including polymers such as silicone rubber, polyurethane, polyethylene, polyvinyl chloride, and latex rubber. An approximate size range of the inner diameter of the access conduits can be 300 μm-1 cm. The access conduits can be integrated into the microfluidic device, or can instead be separate and have attachment points to connect to the microfluidic device.

A pump may regulate blood flow rate into the device, e.g., if arterial blood pressure is not high enough for the particular application or if a venous-venous access is deemed more desirable. In some examples, a physiological blood pressure of 120 mmHg may be sufficient to drive blood flow from an arterial access through the microfluidic device and back to the patient. In other implementations, particularly where veno-venous access is used, a pump is used to drive blood through the microfluidic device. Although optimal pump pressure depends on the desired blood flow, pump pressures ranging from 0-300 mmHg are representative.

The microfluidic device may optionally comprise a reservoir for gas storage. In certain embodiments, the reservoir is an extension of at least one gas channel. In certain embodiments, the reservoir contains oxygen.

The configuration of the fluid removal component is very similar to the polymer layers of the ECMO stack, except the gas permeable transfer membrane is replaced by a fluid-permeable filtration membrane. Example of fluid permeable filtration membranes include microporous polyethersulfone membranes, for example, 0.03 μm pore size polyethersulfone membranes available from Sterlitech, Auburn, Wash. The dimensions of the blood and fluid channels of the fluid removal component may be with in the same ranges as described above for the blood channels of the blood oxygenation/CO₂ removal components. In other embodiments, the dimensions of the blood and fluid channels of the fluid removal component may be dissimilar. The fluid (filtrate) channels may have a greater depth than the blood channels. The fluid channels may have depths of about twice or more than the depths of the blood channels, for example, the fluid channels may have depths of about 140 μm while the blood channels have depths of about 65 μm. The blood and fluid channels may have similar or same widths, for example, about 800 μm.

While the blood flows typically desired for oxygenation, on the order of 4 L/min, may be achieved in a device with up to 40 microfluidic layers to raises O₂ₛₐₜ from 75% or below to 95% or above, a UF blood flow rate of 100 mL/min may be sufficient for effective fluid balance maintenance in injured patients, and therefore a single layer UF device can be seamlessly integrated with the oxygenator (ECMO) stack. In some examples the fluid removal device is placed in the oxygenator layer stack with a shared blood inlet, and the blood manifold will deliver blood to the inlet of the UF layer in parallel with the oxygenator function. The blood outlet of the UF layer will flow back into the outlet manifold, and the filtrate layer will have an outlet that drains to a collection vessel for the removed fluid. The rate of fluid removal may be adjustable to respond to the fluid overload status using a manual or automated valve that may adjust the transmembrane pressure (TMP), so that the fluid removal rate may range between 1 L/day or less and 7 L/day or could be completely shut off if the valve is closed. In some examples, the microfluidic device may include more than one UF device adhered to the ECMO stack.

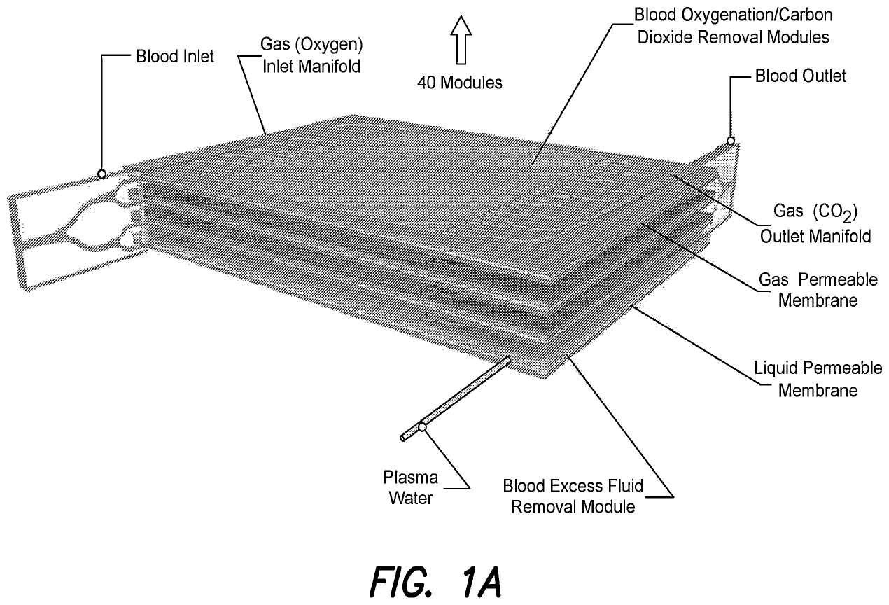

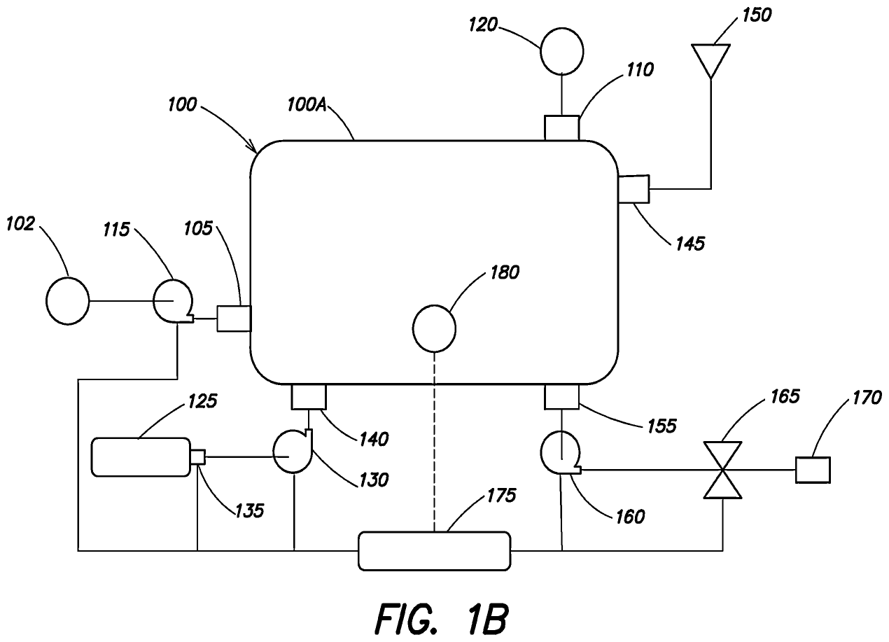

The module stack and blood inlet and outlet manifolds for one example of a device including capabilities for both blood oxygenation/CO₂ removal and excess fluid removal is illustrated in FIG. 1A. The device may include, for example, about 40 units of polymer layer pairs for blood oxygenation/CO₂ removal (ECMO fluidic units) integrated with one or more units of polymer layer pairs for blood excess fluid removal (UF units). Blood inlet and outlet manifolds may supply blood to be treated and collect treated blood from each of the blood oxygenation/CO₂ removal and excess fluid removal unit(s). In some examples, the device is capable of providing for 4 L/min or more of blood flow and UF removal of up to 7 L/day of excess fluid. Although not visible in FIG. 1A, the UF unit includes a fluid collection manifold with a similar configuration as the gas outlet manifold and in fluid communication with the plasma water outlet.

In one example, as illustrated in the block diagram of FIG. 1B, the module stack 100, including a plurality of ECMO fluidic units and at least one UF unit includes a blood inlet 105, for example, a blood inlet manifold as illustrated in FIG. 1A and a blood outlet 110, for example, a blood outlet manifold as illustrated in FIG. 1A. A pump 115 may pump blood from a source of blood 102, for example, a patient or a storage vessel, into the blood inlet 105 and through the plurality of ECMO fluidic units and at least one UF unit. The treated (oxygenated/carbon dioxide reduced/excess fluid reduced) blood exits the module stack 100 at the blood outlet 110 and is directed to a receiver 120 for the treated blood, for example, back into the patient or into a storage vessel. The blood inlet manifold 105 and the outlet manifold 110 are configured to introduce and receive blood from each of the polymer layers of the ECMO through which blood flows during operation without causing substantial damage to the blood. For example, both the blood inlet manifold 105 and the blood outlet manifold 110 may include gradual curving channels rather than right angles. In some implementations, the channels within the manifolds mimic vascular channels. For example, the channels may split at bifurcations. After a bifurcation the size of the channel may be reduced according to Murray's Law.

An oxygen-containing gas, for example, air or pure oxygen is provided from a source of oxygen-containing gas 125, optionally through pump 130 and/or regulating valve/pressure regulator/flow controller 135, to the gas inlet 140, which may be fluidically connected to a gas inlet manifold such as illustrated in FIG. 1A. In some implementations, the pressure regulator 135 includes pressure sensors that send pressure readings to a controller 175 enabling a closed loop control of the pressure within the ECMO fluidic units. In some implementations, the pressure regulator 135 includes a pressure release valve that prevents build-up of pressure substantially beyond a predetermined pressure. For example, the pressure regulator 135 may by a pressure valve that automatically opens when the pressure within the ECMO fluidic units reaches 0.5 atm. The gas passes through the module stack 100 in which oxygen is transferred into blood in the module stack 100 while carbon dioxide diffuses out of blood into the gas stream in the module stack 100 and exits the module stack through the gas outlet 145, which may be fluidically connected to a gas outlet manifold such as illustrated in FIG. 1A. The gas outlet 145 may include a pressure regulator to maintain a desired gas pressure within the gas channels of the device. Gas exiting the gas outlet may be vented to atmosphere through a vent 150.

The module stack 100 further includes a fluid outlet 155 in fluid communication with a fluid collection side of the blood excess fluid removal unit (UF unit) of the module stack 100. Fluid may be drawn out of the fluid collection side of the UF unit through the fluid outlet 155 by a fluid pump/flow meter 160 and optionally through a valve 165, either or both of which may be used to control the transmembrane pressure across the fluid permeable membrane in the UF unit(s). The transmembrane pressure across the fluid permeable membrane may be maintained at a level sufficient to draw up to seven L/day of fluid from out of the blood channels and into the collection channels of the UF unit(s), for example up to about 60 mm Hg. The removed fluid may be sent for drain, collection, or disposal 170. The system may include a computerized controller 175 that regulates the speed of the pumps and controls the gas valve 135 and fluid valve 165. In some examples, a transmembrane pressure sensor 180 is included in the UF unit(s) and provides input to the computerized controller 175 that the computerized controller 175 uses to determine how and when to adjust the fluid pump 160 and/or valve 165. The controller 175 may utilize readings regarding fluid flow rate out of the UF units from the fluid pump/flow meter 160 and/or readings from the transmembrane pressure sensor 180 as inputs to determine how to adjust the fluid pump 160 and/or valve 165 to achieve a desired fluid removal rate.

The module stack 100 may be disposed with a housing 100A including at least the plurality of ECMO fluidic units and the at least one UF unit. The housing 100A may be a pressure resistant housing that includes a hard shell configured to withstand elevated pressures. The housing may be manufactured from a gas and liquid impermeable plastic, such as polycarbonate, or a metal.

It should be appreciated that in other examples suction pumps for blood and/or gas may be fluidically coupled to the blood outlet 110 and/or gas outlet 145 alternatively or in addition to the pumps connected to the blood inlet 105 and/or gas inlet 140.

Examples of the blood treatment device disclosed herein may be fabricated with blood channel networks that vary smoothly in both width and depth across the entire flow circuit. A process for manufacturing examples of the devices disclosed herein may utilize precision CNC-machined metal master molds with microchannel networks that vary smoothly along both the width and depth dimensions. In other examples molds for the polymer layers of the disclosed microfluidic devices may be created through microfabrication, for example, with photopatterned photoresist; however, etched silicon, cured epoxy, and/or electroformed metal can also be used. Master molds comprise ridged features that serve as the inverse of the channel features; when silicone layers are cast into the ridges, smoothly varying microchannel networks are realized. This enables blood flow streamlines with minimal variation in shear and shear gradients.

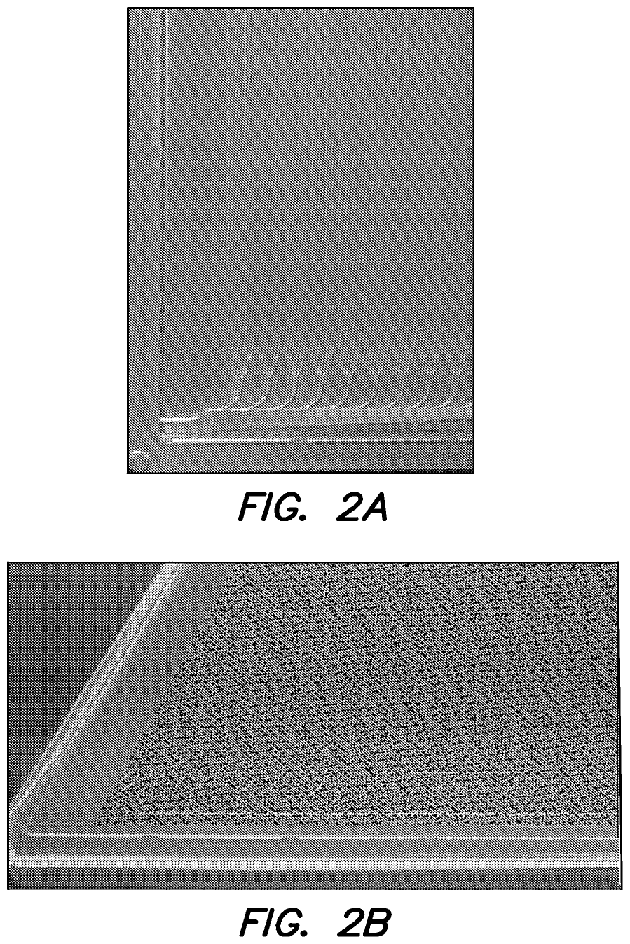

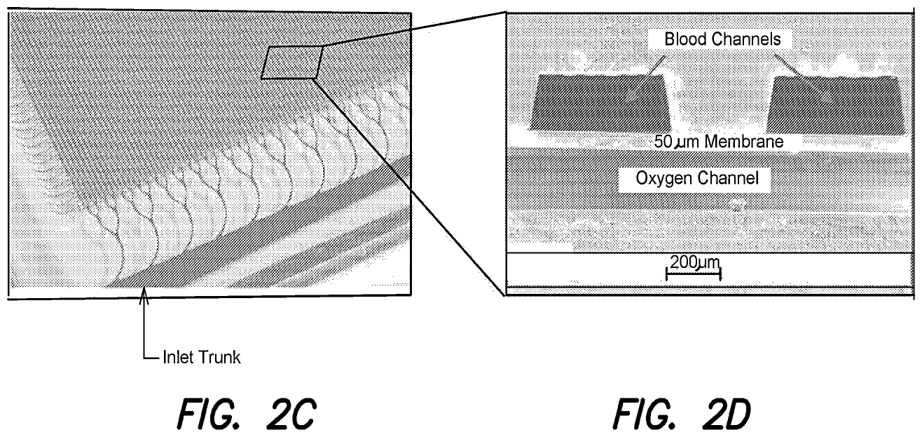

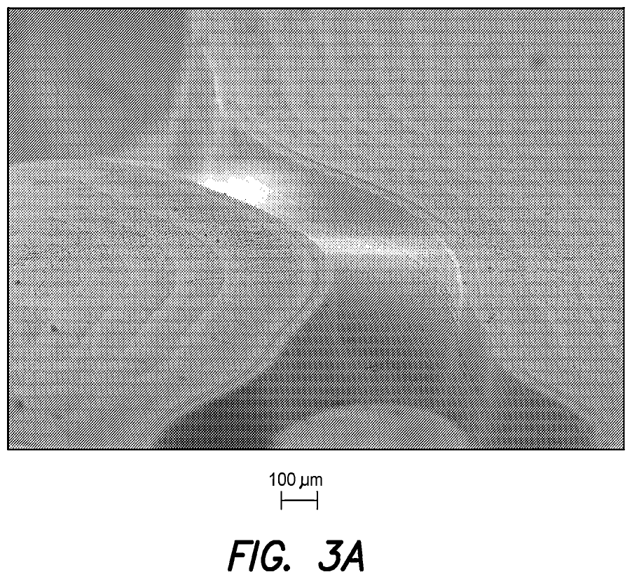

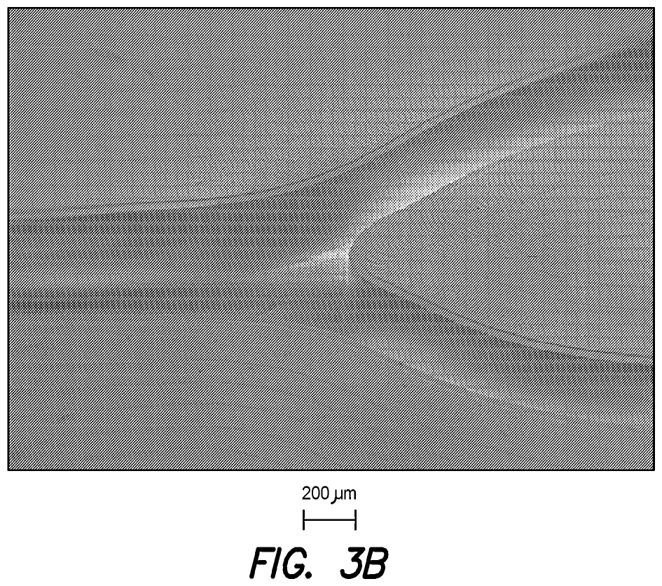

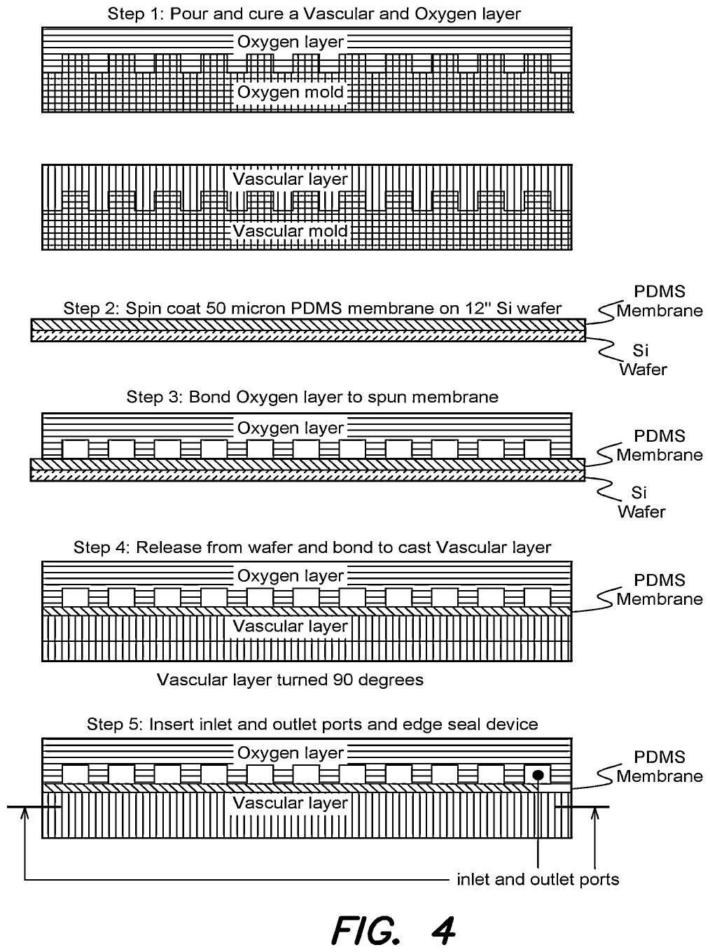

FIGS. 2A-2D illustrate the polymer layer fabrication process in stepwise fashion, where FIG. 2A shows the CNC-machined master mold, FIG. 2B illustrates a cast silicone layer in the mold, FIG. 2C illustrates an assembled oxygenator layer, and FIG. 2D illustrates an SEM cross-section of the oxygen channel-membrane-blood channel sandwich structure. FIG. 3A shows an SEM of a junction region where the channel depth varies smoothly, and FIG. 3B provides an SEM of a smoothly branching channel junction region.

FIG. 4 illustrates the fabrication process for the ECMO fluidic units, where blood and oxygen channel layers are cast against their respective master molds, and silicone gas transfer membranes are spin cast upon polished surfaces, for example, on a silicon wafer, and bonded to the patterned layers to create a sandwich structure that comprises a single layer gas transfer unit for a stacked microfluidic oxygenator. Tubing connections for each layer exit from the left and right (oxygen) or front and back (vascular). Single layers are integrated into a multilayer stack of units utilizing polymer bonding techniques known in the art, for example, as disclosed in U.S. Pat. Nos. 8,647,410, 9,067,179, or 9,717,835, incorporated herein by reference, for example, plasma activation bonding, adhesive bonding, and/or mechanical clamping. In some examples, a hemocompatible surface functionalized moiety or an anticoagulant coating, for example, a heparin-based coating such as CHC™ heparin conjugate from Corline (Sweden) may be applied to all surfaces of the device which blood would contact.

Polymer layers containing fluid and gas channels may be aligned using techniques that include visual alignment and/or alignment via mechanical locating devices. Visual alignment may be achieved by using alignment marks or fiducials integrated into the layers that serve to guide layer alignment for assembly. The alignment may involve magnifying the view of the device layers and any alignment marks, a mechanism to move the layers relative to each other very precisely, and a means of bonding the layers together either permanently or temporarily. In one example, the alignment process is similar to the alignment of a mask to a patterned silicon wafer as used in typical microfabrication processes, yet permits the alignment and bonding of polymer layers. Alignment via mechanical locating devices may be achieved by using specific locking elements integrated into the layers, such that the locating devices on the top of a first layer align and lock with the locating devices on the bottom of a second layer directly above the first layer. In one example, the mechanical locating devices require very little active alignment, as they provide specific and precise location of one layer with respect to another layer.

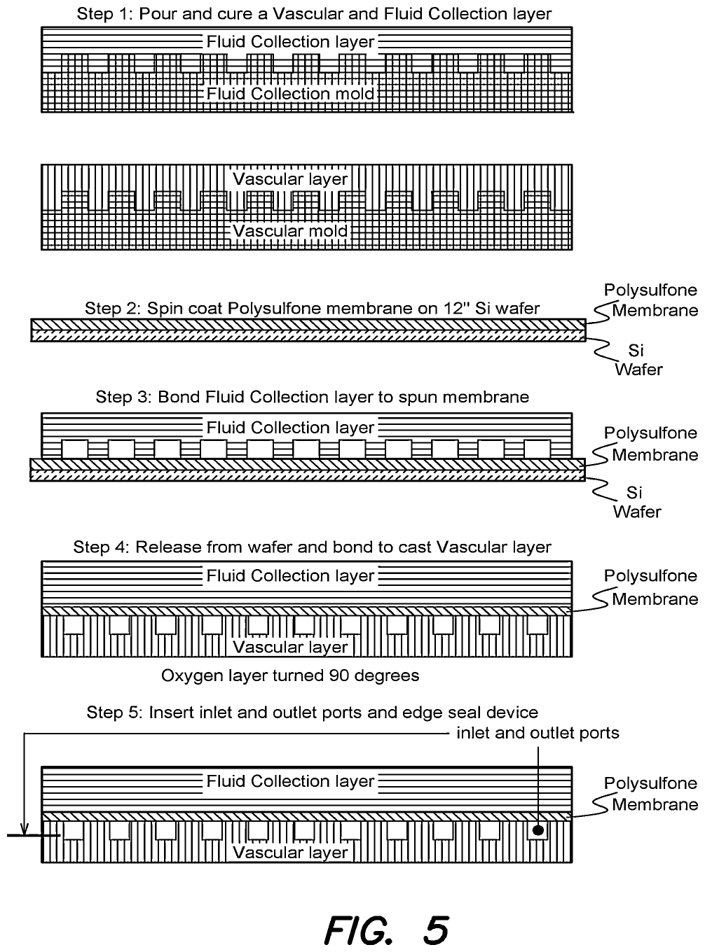

The UF units may be formed in a similar manner as the ECMO fluidic units. As illustrated in FIG. 5, the fluid collection and blood channel layers are cast against their respective master molds. A fluid permeable membrane formed of, for example, polysulfone may be spin cast on upon a polished surface, for example, on a silicon wafer, and bonded to the patterned layers to create a sandwich structure that comprises a single liquid transfer unit for a stacked microfluidic oxygenator. Alternatively, the polysulfone membrane may be obtained premade from a commercial source. Tubing connections for each layer exit from the left (fluid) or front and back (vascular). A single UF unit layer may be integrated into a multilayer stack including a plurality of ECMO fluidic units utilizing polymer bonding techniques known in the art. In some examples, a hemocompatible surface functionalized moiety or an anticoagulant coating, for example, a heparin-based coating such as CHC™ heparin conjugate from Corline (Sweden) may be applied to all surfaces of the UF unit(s) which blood would contact.

In some examples, each or any of the blood, oxygen, and fluid channels may have widths ranging from about 100 μm to about 1000 μm, from about 300 μm to about 800 μm, or from about 500 μm to about 600 μm. In some examples, each of the blood, oxygen, and fluid channels may have heights ranging from about 40 μm to about 250 μm, from about 100 inn to about 200 μm, or from about 100 μm to about 150 μm. In some examples, each or any of the blood, oxygen, and fluid channels may have substantially rectangular, substantially oval, or substantially semi-circular cross sections.

In some examples, the blood, oxygen, and fluid collection layers of the device may be composed of PDMS (for example, NuSil MED-6015, NuSil, Carpinteria, Calif.) from a kit comprising a silicone elastomer and curing agent, which is mixed at a 10:1 ratio by weight using an automated mixer (for example, SpeedMixer DAC 600.1 FVZ, FlackTek, Landrum, S.C.). The elastomer and curing agent may be mixed at 1000 RPM for 15 sec and then a second mixing step at 2000 RPM for 60 sec. For each blood layer, 300 g of the resulting PDMS mixture may be deposited on the corresponding micromachined master mold; a mass of 110 g may be used for the oxygen mold. These casted molds may be placed in a desiccator to de-gas the PDMS for at least 45 minutes. Once degassed, all layers may be cured at 65° C. for a minimum of 3 h. Following curing, blood, oxygen, or fluid collection layers may be peeled off of the molds and trimmed to obtain the appropriate layer dimensions, with handling tabs to permit tubing layer insertion.

For the gas transfer membrane, Silpuran® silicone rubber films at a 50 micron thickness may be utilized. These films may be cut to size and bonded to the blood and oxygen layers using a thin layer of adhesive. A thin layer of adhesive (for example, DOWSIL 3140 RTV, Dow Silicone Corporation, Midland, Mich.) may be deposited onto a flat surface, followed by transfer to the oxygen layer using an ink roller. This layer may then be placed in contact with one surface of the thin gas transfer membrane, and the layer-membrane bond cured overnight at 65° C. The same procedure may be used to attach the blood layer to the other side of the membrane. A perpendicular alignment of the blood and oxygen channels in their respective layers may be maintained during the bonding process, so that the oxygen channels effectively cross each of the blood channels in the design. The assembled stacked structure of blood layer—membrane—oxygen layer may be placed in an oven set to 65° C. for at least 1.5 hours.

Individual blood-membrane-oxygen connections may be made with Tygon® tubing (Cole Palmer, Vernon Hills, Ill.) attached to stainless steel tubing (New England Small Tube). A thin layer of uncured PDMS may be applied to tubing interfaces followed by curing at 65° C. for at least 3 h, followed by leak and pressure testing. After leak and pressure testing, fully functional device layers may be stacked and aligned so that the blood inlet and outlet ports are in vertical alignment. The inlet and outlet blood manifolds may be attached to the individual device layers by connecting the previously attached Tygon® tubing to the stainless steel tubes on the manifolds. The oxygen layers may be connected in series such that the outlet from layer 1 is connected to the inlet of layer 2. All connections of the oxygen layers may be completed using Luer lock connectors. Uniform fluid distribution to and from each of the layers of the stack may be accomplished using a 1-to-40 bifurcating vertical manifold. The branching channels may be designed to smoothly split or recombine the flow such that the fluid in every branch experiences the same level of shear stress. Manifolds may be fabricated by precision machining the half-channel geometry into two identical pieces of polycarbonate, which are subsequently thermally bonded to each other in a heated press to create the full lumen geometry. To provide connection points to the manifold, a polycarbonate male Luer fitting may be glued into the 1-port side of the manifold, while 304 stainless steel tubing (New England Small Tube) is glued into each of channels.

A pressure controller, for example, regulating valve 135 of FIG. 1B, may be connected in line between an oxygen tank and the oxygen inlet of device. This controller controls the inlet oxygen flow out of the tank into the device. The oxygen pressure controller may be tuned for a 100 mL/min oxygen flow rate.

In some examples, the fluid removal module may utilize a membrane that provides for the passage of water, optionally including small soluble factors, or other fluid but not blood cells. This membrane may be one that is used for conventional reverse osmosis systems for water filtration. The fluid permeable membrane may, in some examples be a polysulfone membrane with a 100 kDa molecular weight cutoff. In some examples, the membrane may be a single film with a thickness of, for example, between about 10 μm and about 100 μm, or a film with an active layer between about 10 μm and about 30 μm thick and a backing material that could be several hundreds of microns in thickness. Up to 7 L/day fluid removal rate may be achievable with an average TMP of 40 mm Hg across the fluid permeable membrane and a blood flow rate of 100 mL/min. A valve at the filtrate outlet may be utilized to modulate the TMP from 0-40 mm Hg. The maximum 40 mm Hg TMP may be invoked to design the system at the maximum 7 L/day fluid removal rate, and by gradually closing the valve, the rate of fluid removal may drop toward the lower limit of 1 L/day removal. An alternate or additional option would entail installation of a small filtrate pump that can be adjusted to control the transmembrane pressure (TMP) and therefore the rate of fluid removal. The ultrafiltration module of the device may have the same form factor as a single layer oxygenator component, with the only changes being the substitution of a UF membrane and the presence of a fluid outlet for the filtrate chamber and a valve.

### Example

A laboratory demonstration of a Microfluidic Integrated Renal and Respiratory Organ Support (MIRRORS) device as disclosed herein was performed, showing that single microfluidic oxygenator and fluid removal layers can provide blood gas oxygenation and filtrate removal in a single pass, using anticoagulated porcine blood introduced into the stacked device using a syringe pump. Oxygen transfer and filtrate removal were measured as a function of the blood flow rate, providing independent measures of each component of the MIRRORS function. A valve was inserted in the RRT line to control the transmembrane pressure (TMP) across the RRT device, thereby providing an independent degree of control over the instantaneous ultrafiltration rate (UFR.) It was determined that this tuning feature can be readily used to achieve control necessary to address fluid overload (FO), enabling tunability in a fixed configuration cartridge.

### Computational Modeling:

Extensive reports in the literature address computational modeling of blood gas transfer (See, e.g., J. A. Potkay, M. Magnetta, A. Vinson, and B. Cmolik, “Bio-inspired, efficient, artificial lung employing air as the ventilating gas,” Lab Chip, 2011, doi: 10.1039/c11c20020h.) Here we focus on modeling the UFR versus TMP, to demonstrate the renal replacement function and tunability of the MIRRORS device. A linear relationship between transmembrane pressure and ultrafiltration rate that then bends and reaches a plateau at a threshold value has been reported elsewhere (See, e.g., W. F. Blatt, A. Dravid, A. S. Michaels, and L. Nelsen, “Solute Polarization and Cake Formation in Membrane Ultrafiltration: Causes, Consequences, and Control Techniques BT—Membrane Science and Technology: Industrial, Biological, and Waste Treatment Processes,” J. E. Flinn, Ed. Boston, Mass.: Springer US, 1970, pp. 47-97.)

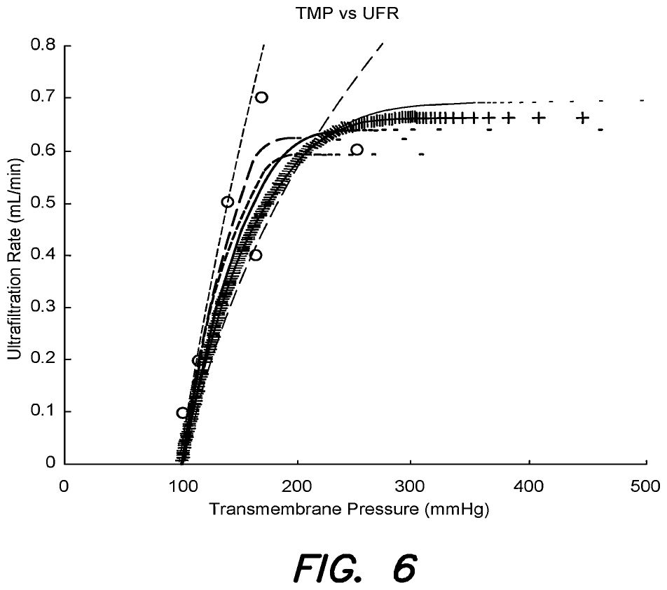

We have collected experimental data as described below where the UFR is varied with a valve and the TMP is measured with a pressure sensor. The experimental data does track with expectations, showing an approximately linear rise of UFR with TMP followed by an apparent leveling off (See data points in circles in FIG. 6). Various best-fit lines to the data using certain assumptions, not detailed herein, are shown along with the experimental data.

### Microfluidic Lung Assist Device Fabrication:

The microfluidic lung assist devices used in these experiments consist of a 50 μm-thick, gas permeable silicone membrane (Silpuran®, Wacker, Munich, Germany) bonded between a microfluidic vascular layer, designed to uniformly distribute blood through a network of channels, and an oxygen layer, designed to carry and transfer oxygen through a parallel array of channels. Each layer is made from a silicone elastomer two-part polymer, Nusil MED-6015 (Avantor, Radnor, Pa.), casted from a micromachined master mold.

### Microfluidic Kidney Assist (Hemofiltration) Device Fabrication:

The RRT component of the device consists of a porous (0.03 μm pore size) Polyethersulfone (PES) membrane (Sterlitech, Auburn, Wash.) between a vascular plate and filtrate plate. Each plate is comprised of a microfluidic channel design that was embossed into a hard plastic template from an aluminum master mold. Both plates include 72 channels that are 15 cm long and 800 μm wide, with a vascular depth of 65 μm and filtrate depth of 140 μm. Assembly of the RRT device was achieved following a 2-step bonding process. First, a thin film of Filterbond R-36 (Hapco, Hanover, Mass.) two-part epoxy was applied to bond the vascular plate to the membrane, then the filtrate plate was bonded to the vascular assembly, followed by weighting and curing for 48 hours. Assemblies of steel tubing, thermoplastic tubing, and a Luer lock fitting were placed into the vascular and filtrate inlet and outlet ports. Epoxy was applied along the device perimeter and was cured for 24 hours before devices were tested for leak paths. The RRT component of the device has an active filtering area of 0.008626 m².

### MIRRORS Assembly and Sample Collection:

One microfluidic oxygenator and one RRT device were connected in a parallel circuit with a split at the front end to distribute venous blood through each layer before rejoining at the outlets for collection of blood and filtrate samples. The blood flow from the syringe was split via tubing between the oxygenator and RRT devices and the resistance of both device lines was calibrated to maintain a consistent 8:1 (lung:kidney) blood flow ratio. A pressurized cylinder of oxygen gas connected to a mass flow controller (Alicat Scientific, Tucson, Ariz.) was used to deliver oxygen to the microfluidic oxygenator. The filtrate outlet was connected via tubing to a syringe on a second syringe pump to collect effluent. Pressure sensors (PendoTECH, Princeton, N.J.) were placed at the vascular inlet and filtrate outlet of the hemofiltration device to monitor pressure over effluent flow rates. Blood was collected from the oxygenator outlet (“arterial”) and the RRT blood outlet (“post-filtration venous”).

### Blood Gas Oxygen Transfer and Ultrafiltration Testing:

Porcine blood (LAMPIRE Biological Laboratories, Inc., Pipersville, Pa.) with Citrate Phosphate Dextrose (CPD) anticoagulant was used in all experiments. Venous blood bags were mixed to obtain a starting oxygen saturation of approximately 65% and were heated to maintain a temperature of 37° C. via a heated rocker (ThermoFisher, Waltham, Mass.). A syringe pump (PHD Ultra, Harvard Apparatus, Holliston, Mass.) with 200 mL syringe supplied a blood flowrate of 20 mL/min. A second syringe pump withdrew filtrate at varied flow rates from the kidney assist device while TMP was being monitored with a pressure sensor. Blood from the oxygenator and RRT device outlet was analyzed via a hemoximeter (Avoximeter 4000, Instrumentation Laboratory, Bedford, Mass.), clinical gas analyzer (GEM Premier 3000, Instrumentation Laboratory, Bedford, Mass.), and CritSpin (Iris Sample Processing, Westwood, Mass.), all measuring tHb (g/dL), O2Hb (%), COHb (%), Met Hb (%), pH, pCO₂ (mmHg), pO₂ (mm Hg), and Hct (%). Data was recorded and analyzed to calculate oxygen transfer performance and TMP.

### Results:

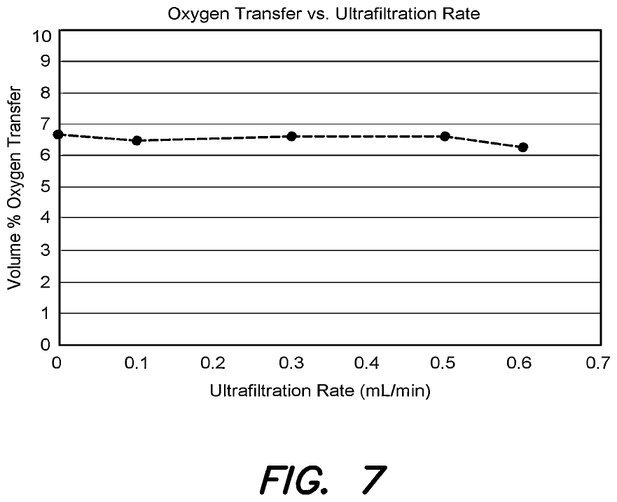

The first result focuses on oxygen transfer in the MIRRORS device across a range of UFR. FIG. 7 shows the volume percent oxygen transfer as a function of the UFR drawn from the RRT device, in which the blood flow rate to the oxygenator was 20 mL/min and the ultrafiltration rate ranged from 0-0.6 mL/min. The vol % transfer remained around 6.5%, indicating that there are no effects on oxygenation resulting from increasing the rate of filtration, as expected.

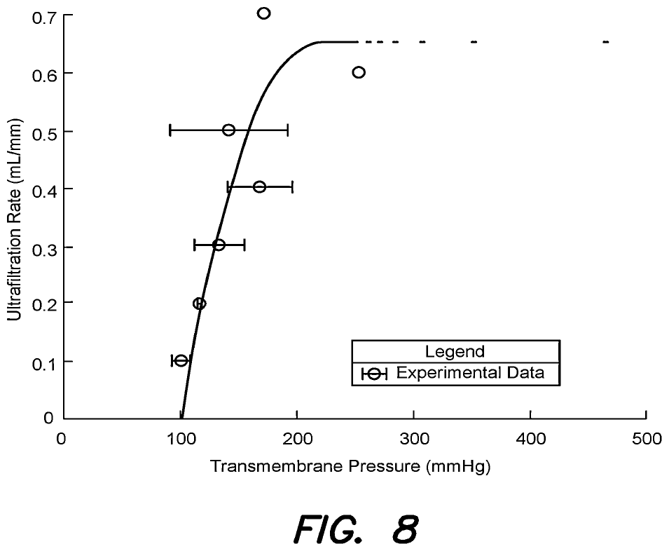

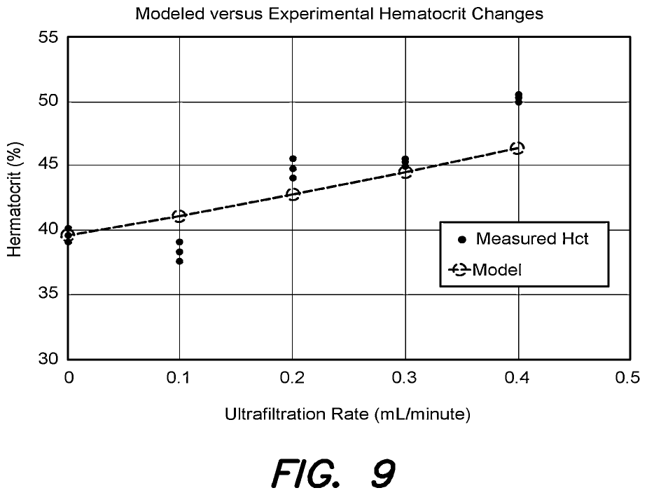

The second result reported here is the relationship between the transmembrane pressure and the ultrafiltration rate in the MIRRORS device. FIG. 8 shows the UFR vs. TMP experimental data from one test device. A best fit curve was calculated. The classic behavior of rapidly rising UFR with TMP up to a threshold where the UFR asymptotically stabilizes is seen in both the model and the data. One other result of note in these studies is the behavior of the hematocrit as the UFR increases. FIG. 9 shows the hematocrit change as a function of UFR, both experimentally measured as described in the Methods, as well as calculated based on concentration of the blood due to plasma water removal. Variability in the hematocrit measurement is likely caused by challenges in obtaining a uniformly mixed sample, but the overall trend is consistent in showing that plasma water removal results in an expected increase in the measured hematocrit in these studies.

### Discussion

Scaling of the small prototype device to clinically relevant levels of oxygenation and fluid removal can be approximated by extrapolating the data obtained on the two-layer MIRRORS prototype. Based on a visual inspection of the best-fit curve, an appropriate range of flow rates to keep the system outside of the plateau region would be 0.4-0.6 mL/min filtrate flow rate from a feed of 2-4 mL/min, giving a filtration fraction of 0.1-0.33. The average Glomerular Filtration Rate of 90-120 mL/min/1.73 m² and the normal feed rate of blood to the kidneys is 1000 mL/min/1.73 m² giving a filtration fraction of 0.09-0.12. Assuming this device can be scaled to support a feed rate of 100 mL/min, only roughly 10 MIRRORS devices running continuously in parallel would be required to replace the kidneys. This rate can be supported within the linear regime which features limited gel layer formation and therefore may be viable for long-term use.

Having thus described several aspects of at least one example, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. An extracorporeal blood treatment module comprising:
a plurality of gas transfer units, each of the gas transfer units including a first polymer layer having a plurality of gas channels, a second polymer layer having a plurality of blood channels, and a gas permeable membrane disposed between the plurality of gas channels and the plurality of blood channels and providing for the transport of gas between the plurality of gas channels and the plurality of blood channels;
a fluid transfer unit adhered to at least one of the plurality of gas transfer units, the fluid transfer unit including a third polymer layer having a plurality of fluid collection channels, a fourth polymer layer having a plurality of blood channels, and a fluid permeable membrane disposed between the plurality of fluid collection channels and the plurality of blood channels and providing for the transport of fluid from the plurality of blood channels into the plurality of fluid collection channels while preventing transport of blood cells from the plurality of blood channels into the plurality of fluid collection channels; and
a housing containing the plurality of gas transfer units and fluid transfer unit.

2. The blood treatment module of claim 1, further comprising a blood inlet manifold having channels fluidically coupled to each of the plurality of blood channels in both the plurality of gas transfer units and the fluid transfer unit.

3. The blood treatment module of claim 2, further comprising a blood exit manifold having channels fluidically coupled to each of the plurality of blood channels in both the plurality of gas transfer units and the fluid transfer unit.

4. The blood treatment module of claim 3, further comprising a fluid exit manifold fluidically coupled to each of the plurality of fluid collection channels in the fluid transfer unit.

5. The blood treatment module of claim 4, further comprising a pump fluidically coupled to the fluid exit manifold and configured to create a transmembrane pressure across the fluid permeable membrane.

6. The blood treatment module of claim 5, further comprising a pressure regulator fluidly coupled to the pump and fluid exit manifold and configured to maintain the transmembrane pressure at a desired level.

7. The blood treatment module of claim 6, further comprising a transmembrane pressure sensor configured to measure the transmembrane pressure.

8. The blood treatment module of claim 7, further comprising a controller configured to receive transmembrane pressure measurements from the transmembrane pressure sensor and adjust operation of one of the pump or the pressure regulator based on the transmembrane pressure measurements.

9. The blood treatment module of claim 1, wherein an upper surface of the third polymer layer is bonded to a lower surface of the second polymer layer of one of the plurality of gas transfer units.

10. The blood treatment module of claim 9, wherein the one of the plurality of gas transfer units is at an end of a stack of the plurality of gas transfer units.

11. The blood treatment module of claim 1, wherein a lower surface of the fourth polymer layer is bonded to an upper surface of the first polymer layer of one of the plurality of gas transfer units.

12. The blood treatment module of claim 11, wherein the one of the plurality of gas transfer units is at an end of a stack of the plurality of gas transfer units.

13. The blood treatment module of claim 1, wherein the plurality of blood channels in each of the plurality of gas transfer units are oriented perpendicular to the plurality of gas channels.

14. The blood treatment module of claim 1, wherein the plurality of blood channels in the fluid transfer unit are oriented perpendicular to the plurality of fluid collection channels.

15. The blood treatment module of claim 1, wherein surfaces of the blood channels in each of the plurality of gas transfer units and in the fluid transfer unit are coated with an anticoagulant.

16. The blood treatment module of claim 1, configured to treat up to seven liters of blood per minute in the plurality of gas transfer units.

17. The blood treatment module of claim 16, configured to raise O₂ₛₐₜ of blood passing through the plurality of gas transfer units from 75% or lower to 95% or higher in a single pass.

18. The blood treatment module of claim 1, configured to treat up to 100 mL of blood per minute in the fluid transfer unit.

19. The blood treatment module of claim 18, configured to remove up to seven liters of fluid per day from blood passing through the fluid transfer unit.

20. The blood treatment module of claim 1, wherein the blood channels of the plurality of gas transfer units are substantially identical in size and shape to the blood channels of the fluid transfer unit.

21. The blood treatment module of claim 1, wherein the gas channels of the plurality of gas transfer units are substantially identical in size and shape to the fluid collection channels of the fluid transfer unit.

22. A blood treatment module comprising:
a first plurality of polymer layers each having a plurality of blood channels; and
a second plurality of polymer layers each having one of a plurality of gas channels or fluid collection channels, each of the first plurality of polymer layers secured to at least one of the second plurality of polymer layers with a membrane, a plurality of the membranes being gas permeable membranes, at least one of the membranes being a fluid permeable membrane.

23. The blood treatment module of claim 22, wherein the fluid permeable membrane is an ultrafiltration membrane.

24. A method of treating blood, the method comprising passing the blood through a blood treatment module comprising:
a plurality of gas transfer units, each of the gas transfer units including a first polymer layer having a plurality of gas channels, a second polymer layer having a plurality of blood channels, and a gas permeable membrane disposed between the plurality of gas channels and the plurality of blood channels and providing for the transport of gas between the plurality of gas channels and the plurality of blood channels;
a fluid transfer unit adhered to at least one of the plurality of gas transfer units, the fluid transfer unit including a third polymer layer having a plurality of fluid collection channels, a fourth polymer layer having a plurality of blood channels, and a fluid permeable membrane disposed between the plurality of fluid collection channels and the plurality of blood channels and providing for the transport of fluid from the plurality of blood channels into the plurality of fluid collection channels while preventing transport of blood cells from the plurality of blood channels into the plurality of fluid collection channels; and
a housing containing the plurality of gas transfer units and fluid transfer unit.
